(19) United States
(12) Patent Application Publication
(10) Pub. No.: US 2009/0098092 A1
(43) Pub. Date: Apr. 16, 2009

(54) Composite Bone Material and Method of Making and Using Same

(76) Inventor: Thomas L. Meredith, (US)

Correspondence Address: WADDEY & PATTERSON, P.C., 1600 DIVISION STREET, SUITE 500, NASHVILLE, TN, 37203, US

(21) Appl. No.: 12/189,755

(22) Filed: Aug. 11, 2008

Related U.S. Application Data
(60) Provisional application No. 60/955,359, filed on Aug. 11, 2007.

(51) Int. Cl. A61K 35/32; C12N 5/06; A61P 19/00

(52) U.S. Cl. 424/93.7; 435/395

(57) ABSTRACT

Bone composite implants and the method of constructing bone composite implants are disclosed which may be used in the repair, replacement, and/or augmentation of various portions of animal or human skeletal systems. Furthermore, the bone composite implants of the present invention may be considered load-bearing implants which are incorporated into the skeletal structure of the patient.

This Utility patent application claims benefit of previously filed provisional patent application No. 60/955,359 filed Aug. 11, 2007 entitled “Human Bone Composite with Ability to Maintain and Release Various Growth Factors.”

### BACKGROUND OF THE INVENTION

1. Technical Field

This invention generally relates to the field of bone composite implants and the method of constructing bone composite implants for the use within a mammalian patient. The bone composite implants or osteoimplants of the present invention may be used in the repair, replacement, and/or augmentation of various portions of animal or human skeletal systems. Furthermore, the bone composite implants of the present invention may be considered load-bearing implants which are improved in being incorporated into the skeletal structure of the patient.

2. Background

The practice of donating and transplanting bone tissue is beginning to form an important part of therapy of ailments involving bone. Generally, bone has a variety of components including lamellae, haversian canals, blood vessels running in connection with the canals, and the marrow portion also having blood vessels extending there into the marrow. Commonly known within medical science is the practice of tissue grafting of live tissue from the same patient including bone grafting where tissue such as bone is removed from one part of the body and inserted into tissue in another part of the same body. In the past, this method has been desirable as the tissue was believed to be highly osteoconductive. With respect to living bone tissue, it was desirable in the past to be able to remove a piece of living tissue graft material which was the exact size and shape needed for the host site though unfortunately this method was very difficult and often unsuccessful.

Until recently, developers of bone transplants and prostheses have believed that it is desirable to maintain graft tissue in a living state during the grafting process. It is relatively undisputed that the use of living tissue in a graft will promote bone healing, but recent surgical experience has shown that healing can be achieved with allografts of non-living bone material which has been processed.

Processing of bone material which does not contain living tissue is becoming more and more important. Non-living bone grafting techniques have been attempted both for autografts and for allografts. The use of autograft bone is where the patient provides the source of the bone, and the use of allograft bone is where another individual of the same species provides the source of the bone.

In the prior art, transplanted bone has been used to provide support, promote healing, fill bony cavities, separate bony elements (such as vertebral bodies), promote fusion (where bones are induced to grow together in a single, solid mass), or stabilize the sites of fractures.

For example, Nashef U.S. Pat. No. 4,678,470 discloses a method of creating bone graft material by machining a block of bone to a particular shape or by pulverizing and milling it. The graft material is then tanned with glutaraldehyde to sterilize it. This process can produce bone plugs of a desired shape.

In the Nashef process, the process of pulverizing or milling the bone material destroys the structure of the bone tissue. The step of tanning it with glutaraldehyde then renders the graft material completely sterile.

It is now possible to obtain allograft bone which has been processed to remove all living material which could present a tissue rejection problem or an infection problem. Such processed material retains much of the mineral quality of the original living bone, rendering it more osteoinductive. Moreover, it can be shaped according to known and new methods to attain enhanced structural behavior. In fact spine surgeons express a distinct preference for such materials, and at least one supplier, the Musculoskeletal Transplant Foundation (MTF), has introduced femoral ring allografts for spine surgeries.

Research shows that such allografts are very favorable for spinal surgery. According to Brantigan, J. W., Cunningham, B. W., Warden, K., McAfee, P. C., and Steffee, A. D., A compression Strength of Donor Bone for Posterior Lumbar Interbody Fusion, Spine, Vol. 18, No. 9, pp. 12113 21 (July 1993):

Many authors have viewed donor bone as the equivalent of autologous bone. Nasca, et al. compared spinal fusions in 62 patients with autologous bone and 90 patients with cryopreserved bone and found successful arthrodesis in 87% of autologous and 86.6% of allograft patients. (Citations omitted.).

A drawback of fabricating transplants and prostheses from donated allograft is that the process necessitates discard of a great deal of scrap and powdered bone material. Good quality donated bone is a scarce resource, so that devising a method of using scrap and powdered allograft bone material would be of great assistance to this highly beneficial endeavor. The present invention uses ground bone to make solid shapes. The results of the present invention are superior to the prior art processes and the process and composite of the present invention allows for a greater amount of donor bone to become available. With a transplanted allograft, older bone may be too brittle and weak.

In the fabrication of bone transplants, it was observed that bone material which yields to compressive loads at the exterior surfaces without significant degradation of the interior structural properties, such as cancellous or trabecular bone, can be shaped. It is not unusual that reshaping of a graft tissue is necessary to obtain the best possible graft. In particular, bone tissue may be stronger and better able to bear force when it is denser and more compact.

Additionally, prior art techniques have a serious limitation in that bone parts and bone products made from allograft cortical tissue may be limited in size, dimension and shape because of the anatomical limits on the thickness and length of the source bone. With the method of the present invention, many shapes and forms can be fabricated from allograft cortical bone tissue including pins, screws, plates, intervertebral discs, and the like for use in surgery.

Allograft bone occurs in two basic forms: cancellous bone (also referred to as trabecular bone) and cortical bone. Cortical bone is highly dense and has a compound structure comprised of calcium hydroxyapatite reinforced with collagen fiber.

Compression of allograft bone is desirable from general considerations. Generally, bone samples are stronger when they are denser. Compressing allograft bone increases its density and thus generally strengthens the allograft. In addition, recent studies have indicated that the shell of vertebral bone is very much like condensed trabecular bone. Mosekilde, L., A Vertebral structure and strength in vivo and in vitro, Calc. Tissue Int. 1993; 53 (Suppl):121 6; Silva, M. J., Wang, C., Keaveny, T. M., and Hayes, W. C., A Direct and computed tomography thickness measurements of the human lumbar vertebral shell and endplate, Bone 1994:15:409 14; Vesterby, A., Mosekilde, L., Gunderson, H. J. G., et al., Biologically meaningful determinants of the in vitro strength of lumbar vertebrae, Bone 1991; 12:219 24.

Compression also allows conversion of larger irregular shapes into the desirable smaller shape, thereby permitting more disparate sources of allograft bone to be used. By compressing bone to a given shape it is possible to configure the allograft to match a preformed donee site prepared by using a shaped cutter to cut a precisely matching cut space. In particular, this method of formation facilitates the formation of match mated surfaces of the implant for the formation of a particular shape for skeletal repair or revision.

For the reasons stated above, in certain embodiments of the present invention, compression is useful as part of the molding step in forming the substrate bone composites of the present invention. However, an advantage of the present invention is that in some embodiments compression is not required, and in some embodiments it is preferred—but at very low pressure when compared to the compression levels of the prior art.

It is known that allograft bone can be reshaped into one of many configurations for use as an implant. Various methods, including that of Bonutti, U.S. Pat. Nos. 5,662,710 and 5,545,222, can be used to shape allograft material into the desired shape.

A goal of a bone composite transplant is that the transplant is readily received and hosted by the receiving mammal, with bone fusion occurring (i.e., the composite should be biocompatible and osteoinductive). Today, the only other osteoinductive implants are allograft shapes that have been cut and shaped from cadaver donated bone. This method has serious drawbacks in that it is difficult for sufficient fusion to take place and the implant usually lacks sufficient structural strength and density.

U.S. Pat. No. 6,025,538 to Yaccarino, III, discloses allograft bone devices for surgical implantation in the bone tissue.

U.S. Pat. No. 5,439,864 to Rosin et al., discloses shaped demineralized bone for use in the surgical repair of bone defects.

U.S. Pat. No. 5,662,710 to Bonutti, discloses a tissue press for shaping or compressing a piece of graft tissue.

U.S. Pat. No. 5,899,939 to Boyce et al. discloses a bone-derived implant that comprises cortical bone and is used to repair, replace, or augment various portions of animal and human skeletal systems. The bone implant of this invention is made up as individual layers that may be held together by adhesives. Finally, the bone-derived implant of this invention may have one or more cavities which may be filed with demineralized bone powder. This patent fails to disclose making an implant or prosthesis from ground bone powder.

U.S. Pat. No. 6,025,538 to Yaccarino, III discloses allograft bone devices for surgical implantation in the bone tissue. The device is larger than the natural dimensions of a cortical bone layer and is made by combining two or more smaller pieces to form a compound bone structure. A pin may be placed through the component bone members of the bone structure. Finally, each bone member is shaped to form a groove to receive the end of the other bone member. The device of this invention may be processed to form compound bone pins, bone screws, plates, disks, wedges, blocks, etc. The devices may be secured together by using any surgical bone adhesive with a synthetic absorbable or non-absorbable polymer in connection with the pin that connects the two bone pieces together.

U.S. Pat. No. 6,090,998 to Grooms et al. discloses a unitary bone implant having at least one rigid, mineralized bone segment. The implant may be machined to include threads, grooves, etc. to provide a means for fixation of the implant directly to a bone machined in a complimentary fashion. The implant of this invention may be used to repair or replace ligaments, tendons, and joints.

U.S. Pat. No. 6,045,554 to Grooms et al. discloses an interference screw manufactured from cortical allograft bone tissue may be used as a fixation screw for cruciate ligament graphs. The screw is made by obtaining a fragment of bone from the cortex and machining the thread, tip and drive head of the screw. More specifically, the section is removed from a femur or tibia, a dowel of the tissue is machined. The machining may be done by a grinding wheel.

U.S. Pat. No. 5,507,813 to Dowd et al. discloses a process for making surgically implantable materials fabricated from elongate bone particles. The particles may be graded into different sizes. Additionally, the particles are described as filaments, fibers, threads, slender or narrow strips, etc. The elongate bone particles may be mixed with an adhesive and/or filler. The fillers include bone powder.

U.S. Pat. No. 5,061,286 to Lyle discloses an osteoprosthetic implant with demineralized bone powder attached thereto. The bone powder apparently provides an osteogenic coating for the prosthesis. This coating allows the prosthesis to be firmly anchored to the bone repair site. The prosthesis device may be polymeric. The bone particles may be adhere to the prosthetic device and each other by a binder. Cyanoacrylate is disclosed as one of the binders.

U.S. Pat. No. 5,516,532 to Atala et al. discloses a method of making a cartilage and bone preparation using ground bone. The ground bone is apparently mixed with polymeric carriers and provides a suspension that may be injectable and used for correction of a variety of tissue defects. The suspension is typically injected through a cystoscopic needle or via a syringe directly into a specific area where the bulking is required.

U.S. Pat. No. 6,136,029 to Johnson et al. discloses an open-celled article that is useful as a bone substitute material that is highly porous and is of low density. The article comprises a framework that is preferably ceramic.

U.S. Pat. No. 6,294,187 to Boyce, et al. discloses an osteoimplant for use in the repair, replacement, and/or augmentation of various portions of animal or human skeletal systems. The implant of this patent comprises bone particles in combination with one or more biocompatible components. The implant is made by applying compressive force of at least 1,000 psi to the composition.

U.S. Pat. No. 5,565,502 to Glimcher, et al. discloses a process for removing and isolating the calcium-phosphate crystals of bone. The bone powder is prepared by milling bone in liquid nitrogen and sieving to a particle size ranging up to approximately 20 microns. The bone particles are then suspended in an organic solvent. The purified calcium-phosphate crystals are isolated from the bone and are useful as an aid to induce and promote bone healing.

U.S. Pat. No. 5,824,078 to Nelson, et al. discloses an allograft bone press. The bone press is used to compress cancellous bone chips to conform to a shape of a mold.

U.S. Pat. No. 4,645,503 to Lin, et al. discloses moldable bone-implant material. This material is prepared by mixing hard bone-graft filler particles with a biocompatible thermoplastic binder.

U.S. Pat. No. 4,843,112 to Gerhart, et al. discloses a moldable, biocompatible, polyester-particulate composite that can be used for reinforcement of fractures in a bone. This invention is directed to a biodegradable cement composition adapted for use in the surgical repair of living bone and for the controlled-released delivery of pharmaceutical agents.

U.S. Pat. No. 6,132,472 to Bonutti discloses a tissue press for shaping or compressing a piece of tissue. This apparatus and method is designed to press or shape tissue while preserving the tissue alive.

In response to the need for a composite material to make use of bone fragments and bone power when fabricating implants and prosthetic devices for bone, the current inventor developed a method of forming a bone composite as disclosed in U.S. Pat. No. 7,001,551. In the '551 patent, a method of forming a bone composite was disclosed comprising: providing bone tissue; grinding said bone tissue to form ground tissue; transferring the ground bone tissue into a mold; applying a binder to the bone tissue; applying a vacuum to the mold; and optionally milling or refining the bone composite to the desired shape.

Another embodiment of the '551 patent includes a method of forming a bone composite, comprising: (i) providing bone tissue; (ii) grinding said bone tissue to form ground bone tissue ranging in size from about 125 microns to about 1000 microns; (iii) transferring said ground bone tissue into a mold; (iv) applying a cyanoacrylate binder to the bone tissue; (v) applying a vacuum to the mold; (vi) applying a compressive force of less than 1000 psi to the mold; and (vii) optionally milling or refining the bone composite to the desired shape.

Typically, the bone composite as produced and embodied in the '551 patent, comprises a composite which is osteoinductive comprising ground bone tissue molded to form a desired shape and a cyanoacrylate binder. Additionally, the bone composite of the '551 patent comprises random voids which also aid in osteoconductivity.

In response to the need and desire of creating a bone composite with an even greater level of osteoinductivity, the current inventor developed the present invention for an improved bone composite and method of making the same which induces an even faster build up of new mammalian bone cells in relation to the bone composite implanted within the patient.

### BRIEF SUMMARY OF THE INVENTION

An object of the present invention is therefore a method of producing a bone tissue composite that has improved osteoinductivity which increases the rate at which structural bone fusion will occur within a patient. Furthermore, such bone tissue composite may also have excellent strength characteristics including an excellent load-bearing ability.

Another object of the current invention is to provide a composite material utilizing bone powder and/or fragments combined with at least one growth accelerator as well as a method to manufacture and shape the composite, including the growth accelerator, into usable implants and/or bone prostheses. In preferred embodiments of the present invention, bone composites formed from the method of the present invention may have a variety of different strength and density characteristics controlled via the formation of the composite so that the composite which may include a growth accelerator may be utilized in a variety of different applications.

Another object of the present invention is to provide a method which enables a bone composite to include a growth accelerator so that upon implantation into a patient, structural bone fusion will occur more rapidly.

Furthermore, it is an object of the present invention to provide a bone composite containing a growth accelerator which may be readily received and hosted when received by another mammal. The composite of the present invention with the included growth accelerator provides for more rapid bone fusion, and more specifically, the biocompatible and osteoinductive process allows the body to lay down native bone in combination with the implanted bone composite with included growth factor.

More specifically, the present invention relates to a method of forming a bone composite including a growth accelerator comprising: providing bone tissue; grinding said bone tissue to form ground tissue; transferring the ground bone tissue into a mold; applying a binder to the bone tissue; applying a vacuum to the mold; optionally applying a longer duration of vacuum; injecting a carrier having a growth accelerator into the bone composite; optionally applying pressure or vacuum to the bone composite with the carrier and growth accelerator; and removing the bone composite containing a carrier with growth accelerator after a predetermined amount of time has occurred. Preferably, the bone tissue utilized in the bone composite is substantially cortical bone tissue (i.e., greater than about 40% to about 50%) and preferably, the bone tissue is substantially demineralized (i.e., greater than about 40% to about 50%).

The carrier utilized in the present invention is any sort of polymeric material, liquid, or chemical compound which may be utilized to transport the growth accelerator into the bone composite. Preferably, the carrier is a bioabsorbable polymer which may include a bioabsorbable cyanoacrylate alone or in combination with a variety of other polymers for the transport of the growth accelerator into the bone composite. Additionally, the carrier may include a variety of different ionic charges and/or a predetermined magnetic charge so that the growth accelerator may more rapidly induce bone fusion to occur in or about the bone composite. Advantageously, the cyanoacrylate when used as a carrier may also function as a binder for the bone composite material. As such, rapid polymerization of the cyanoacrylate within the bone composite material will occur upon exposure to moisture within a patient thus providing for substantial rigidity of the composite material within a short period of time.

Bone tissue for use in the bone composite material of the present invention includes some cortical bone tissue and preferably includes greater than about 50% cortical bone tissue, more preferably in the range of greater than about 50%-70% cortical bone tissue, more preferably in the range of greater than about 50%-70% cortical bone tissue, more preferably in the range of greater than about 50-95% cortical bone tissue, more preferably 90% cortical bone tissue, and more preferably greater than about 95% cortical bone tissue. The size of the ground bone particles can vary, but typically the particles will range in size from 125 to 1000 microns in size.

The growth accelerator provided into the bone composite may be any type of growth accelerator which increases or induces bone fusion with the bone composite to occur more rapidly within a patient. Preferably, the growth accelerator may be a transforming growth factor (TGF) which may include bone, morphogenetic proteins (BMPs) so that the formation of bone is induced and occurs more rapidly. Furthermore, the growth accelerator may also include both artificial and nature TGFs as well as other chemicals known to provide for an increase in the rate of bone growth.

Another embodiment of the present invention is a method of forming a bone composite comprising inserting growth accelerators into a bone composite so that any bone composite may be improved and more rapidly increase bone formation when implanted within a patient.

A further embodiment of the present invention includes a method of bone fusion comprising inserting a bone composite containing a bioabsorbable carrier with growth accelerator into a patient wherein upon insertion of the bone composite into the patient, the carrier is absorbed with the growth accelerator and bone fusion is thereby accelerated.

These aspects and others that will become apparent to the artisan upon review of the follow description can be accomplished by providing a bone composite material formed from bone tissue and including a growth accelerator into the bone composite. The inventive improved bone composite and method of forming the improved bone composite includes a growth accelerator which provides for an improved fusion of the composite with natural bone from the patient's body.

It is to be understood that both the forgoing general description and the following detailed description provide embodiments of the invention and are intended to provide an overview of framework of understanding to nature and character of the invention as it is claimed.

### DETAILED DESCRIPTION OF THE EMBODIMENTS

The method and composition of the present invention can be used with any mammals, preferably horses and humans, and most preferably humans. As was previously mentioned, the bone composites of the present invention include a growth accelerator to better promote healing in the patient with the implanted novel bone composite.

Preferably, the growth accelerator may comprise one or more transforming growth factors (TGF) which are primarily polypeptide growth factors some of which play crucial roles in tissue regeneration. Often, TGFs are described as two different classes including TGF-alphas and TGF-betas with TGF-betas existing in humans in about three different subtypes. As has been generally studied, TGF-beta ligands include bone morphogenetic proteins (BMPs) which can induce the formation of bone and cartilage within a human. Currently, there are about 16 different BMPs that have been discovered with either single BMPs or a combination of BMPs which may be utilized as the growth accelerator for inclusion within the novel bone composite of the present invention. Furthermore, for purposes of this application, the use of stem cell technology in use with a bone composite may generally be defined as a transforming growth factor in discussing the multiple embodiments of the invention of the application.

More specifically, in any embodiments of the present invention where the growth accelerator may include BMPs, the BMPs may interact with specific receptors to mobilize proteins, and thus, assist in bone development. While a variety of different BMPs exist, most belong to the TGF-beta family of proteins, which may be utilized as growth accelerators. Particularly, BMP-2, BMP-3, BMP-4, BMP-7 and BMP-8a, as well as other BMPs, may increase, induce or otherwise assist in bone formation. Generally, BMP-2 and BMP-7 are considered osteogenic bone morphogenetic proteins with BMP-2 being able to stimulate the production of bone, and BMP-7 being an important component in the transformation of mesenchymal cells as well as being involved in bone homeostasis.

In further embodiments, the growth accelerator of the present invention may include a variety of other artificial components which can be utilized in inducing bone growth. For example, different artificial components may be utilized as the growth accelerator for the stimulation of osteoblasts for forming new bone as well as the manipulation of osteoclasts which typically break down bone tissue. Such artificial growth accelerators may include synthetic hormones, naturally occurring hormones as well as artificial growth factors and also recombinantly created growth factors which may also assist in inducing an accelerated healing of bone about the novel bone composites.

In further embodiments, compounds for the manipulation of hormones such as the parathyroid hormone as well as the development and usage of calcitonin may also be utilized as growth accelerators for the present invention. Additional potential candidates for the growth accelerator include, but are not limited to, hormones such as amylin and adrenonedullin which are somewhat related to calcitonin which may also stimulate the proliferation of osteoblasts so as to build new bone and more rapidly fuse the bone composite implant into the patient's body as well as any artificial or recombinantly created forms of amylin, adreonedullin or other artificial or recombinantly growth factors.

Furthermore, other compounds may be utilized as growth accelerators, and as such, the scope of the present invention is not limited to the specific proteins and hormones discussed within the above-captioned application for growth accelerators though many may be generally described as natural, artificial or recombinant produces of TGFs, BMPs, hormones or chemicals with significant homology to hormones, all of which may be useful in inducing and propagating new bone growth to accelerate the fusion of the novel bone composite of the present invention into the patient's body.

The growth accelerator may be incorporated into a bone composite in a variety of different methods. Preferably, the growth accelerator is in the bone composite in a manner so that upon implantation the growth factor may be absorbed by the body so as to promote localized accelerated growth of bone so that healing occurs rapidly. As such, the growth accelerator is preferably included within the bone composite in a manner so that it may be associated with surrounding tissues and fluids of the body of the patient. In a preferable arrangement, the growth accelerator of the novel bone composite is absorbed out of the bone composite though remains within the bone composite in a stable form prior to implanting the improved composite into the patient. This provides for a quicker and less invasive surgery as the bone composite with growth factor may comprise a pre-packaged sterile bone composite which may thereby eliminate or reduce autograph surgery and thus provide swift healing with a high percentage of surgical success.

Preferably, the growth accelerator is input into the bone composite via a carrier which also may be maintained within the bone composite. While all embodiments do not require a carrier and should not be limited to the inclusion of a carrier, preferably embodiments include a carrier compound for the improved association of a growth accelerator within the bone composite material of the present invention. Most preferably, the carrier material is a bioabsorbable material which can be mixed with the growth accelerator and included within the bone composite.

The carrier may include bioabsorbable cyanoacrylate which may have a predetermined viscosity, weight, and density which can be used to transport and maintain the growth accelerator within the bone composite. One such type of bioabsorbable cyanoacrylate is described in U.S. Pat. No. 6,224,622 issued to Chemence, Inc. which describes bioabsorbable cyanoacrylate-based tissue adhesives which is hereby expressly incorporated by reference in its entirety. Other suitable materials for a carrier for the growth accelerator include bioabsorbable materials which that do not weaken the bone composite or impair the bone composite's structural integrity. Advantageously with the use of cyanoacrylate as a carrier, the cyanoacrylate doubles as the binder thus necessitating less needed components for the bone composite. In further embodiments the materials used as carriers should not produce toxic reactions or create overall harmful effects to the patient. Generally, the bioabsorbable materials suitable as a carrier readily degrade upon the implantation of the novel bone composite within the patient. Such chemical compounds which may also be utilized may include, but are not limited to poly-L-lactide or copolymers thereof, homopolymers of beta-hydroxybutyric acid, polymers of omega hydroxyacids, polyamides, polyanhydrides, polyorthoesters, polyglycolyic acid, polyglactin material, poliglecaprone, absorbable lactomers, and various other biodegradable polymers such as biodegradable aliphatic polyesters, polyester amides, polyanhydrides, and polyphosphazenes.

Upon the selection of a proper and desired carrier, the carrier may then be mixed with a growth accelerator so that the carrier with growth accelerator may be included within a bone composite. Preferably, the carrier and growth accelerator are combined and then inserted within a preformed bone composite so that the carrier-growth accelerator combination is within voids within the bone composite substrates. While not limited to such bone composite substrates, a preferable method of creating the novel bone composite of the present invention is to utilize as a bone composite substrate, the materials created by the method as described in U.S. Pat. No. 7,001,551 issued to the applicant which is hereby incorporated by reference in its entirety. In comprising the bone composite substrate for the inclusion of the carrier and growth accelerator combination, donor bone utilized in the substrate of the composite may be of the same species as the recipient bone for the patient. That is preferably human bone is used in make a bone composite substrate for the novel bone composite that will be used by humans.

Additionally, in preferable substrates, the bone tissue is demineralized. “Demineralized,” as applied to the bone particles used in the substrate bone composite, is intended to cover all bone particles that have had some portion of their original mineral content removed by a demineralization process. The bone particles are optionally demineralized in accordance with known and conventional procedures in order to reduce their inorganic mineral content. Demineralization methods remove the inorganic mineral component of bone by employing acid solutions. Such methods are well known in the art, see for example, Reddi et al., Proc. Nat. Acad. Sci. 69, pp 1601 1605 (1972). The strength of the acid solution, the shape of the bone particles and the duration of the demineralization treatment will determine the extent of demineralization. Reference in this regard may be made to Lewandrowski et al., J Biomed Materials Res, 31, pp 365 372 (1996). Additionally, the bone particles may be demineralized as set forth in U.S. Pat. No. 6,294,187.

As utilized herein and throughout incorporated U.S. Pat. No. 7,001,551, the phrase “superficially demineralized” as applied to the bone particles refers to bone particles possessing at least about 90 weight percent of their original inorganic mineral content. The phrase “partially demineralized” as applied to the bone particles refers to bone particles possessing from about 8 to about 90 weight percent of their original inorganic mineral content, and the phrase “fully demineralized” as applied to the bone particles refers to bone particles possessing less than about 8, preferably less than about 1, weight percent of their original inorganic mineral content. The unmodified term “demineralized” as applied to the bone particles is intended to cover any one or combination of the foregoing types of demineralized bone particles.

The type of mammalian bone that is most plentiful and most preferred as a resource for the composites for the substrate composite is cortical bone, which is also the form of bone tissue with the greatest compressive strength.

The bone tissue is ground or pulverized. Pulverized bone can be collected and separated into a number of batches, each batch comprising a different mean particle size. The particle size can vary from fine to coarse. The properties of the final composite to be produced can be tailored by choice of particle size. For example, particles in the range of from about 125 to about 1000 microns can be used for making bone composites useful for skeletal repair and revision.

The resulting bone powder is placed in a mold and compressed using compression tooling. The measurements of the bone powder (weights and volume) are all predetermined, and one of ordinary skill in the art would understand the measurements to be dependant upon the size and shape of the desired resulting composite to be manufactured.

In a preferred embodiment, the ground bone tissue is hydrated before being placed in the mold. Most preferably, the ground bone tissue is hydrated in an amount of about 1 to about 10% (volume), preferably in an amount of about 1 to about 5%. Preferably the hydrate is non-isotonic water, and is preferably applied by injection, spray bath, or soaking.

The mold may be any commercially mold that has pneumatic or vacuum capabilities. Preferably, the mold is a virgin Teflon™ or polyethylene mold that is contained in a stainless steel envelope. The mold preferably has a stainless steel pneumatic cylinder, vacuum pump, exhaust filtration, and pneumatic silencers.

Typically the input pressure, bore size of the pneumatic cylinder, and vacuum level (inches of Hg based on a standard barometer reading at atmospheric pressure (14.7 psi)) is predetermined and dependent upon the desired size, desired shape, and desired density of the substrate composite.

The mold preferably will incorporate predetermined number of orifices of a predetermined size, to help assure that the substrate composite will receive evenly distributed pneumatic induced pressure and vacuum flow (Pascal's law).

The bone particles of the substrate composite may be combined with one or more of the biocompatible components set forth in U.S. Pat. No. 6,294,187, incorporated herein by reference. That is, the substrate composite may be combined with one or more biocompatible components such as wetting agents, biocompatible binders, fillers, fibers, plasticizers, biostatic/biocidal agents, surface active agents, bioactive agents, and the like, prior to, during, or after compressing the bone particle-containing composition. One or more of such components can be combined with the bone particles by any suitable means, e.g., by soaking or immersing the bone particles in a solution or dispersion of the desired component, by physically admixing the bone particles and the desired component, and the like.

At least a binder is applied to the bone powder. The binder may be applied by an injection, spray, bath, soaking, or layering. Preferably the binder is applied to the bone tissue in the mold, and preferably during a period while the mold is under vacuum. The binder should be biocompatible. Preferably the binder is a cyanoacrylate.

Suitable wetting agents include biocompatible liquids such as water, organic protic solvent, aqueous solution such as physiological saline, concentrated saline solutions, sugar solutions, ionic solutions of any kind, and liquid polyhydroxy compounds such as glycerol and glycerol esters, and mixtures thereof. The use of wetting agents in general is preferred in the practice of the substrate composite, as they improve handling of bone particles. When employed, wetting agents will typically represent from about 20 to about 80 weight percent of the bone particle-containing composition, calculated prior to compression of the composition. Certain wetting agents such as water can be advantageously removed from the osteoimplant, e.g., by heating and lyophilizing the osteoimplant.

Suitable biocompatible binders include biological adhesives such as fibrin glue, fibrinogen, thrombin, mussel adhesive protein, silk, elastin, collagen, casein, gelatin, albumin, keratin, chitin or chitosan; cyanoacrylates; epoxy-based compounds; dental resin sealants; bioactive glass ceramics (such as apatite-wollastonite), dental resin cements; glass ionomer cements (such as Lonocap® and Inocem® available from lonos Medizinische Produkte GmbH, Greisberg, Germany); gelatin-resorcinol-formaldehyde glues; collagen-based glues; cellulosics such as ethyl cellulose; bioabsorbable polymers such as starches, polylactic acid, polyglycolic acid, polylactic-co-glycolic acid, polydioxanone, polycaprolactone, polycarbonates, polyorthoesters, polyamino acids, polyanhydrides, polyhydroxybutyrate, polyhyroxyvalyrate, poly (propylene glycol-co-fumaric acid), tyrosine-based polycarbonates, pharmaceutical tablet binders (such as Eudragit® binders available from Huls America, Inc.), polyvinylpyrrolidone, cellulose, ethyl cellulose, micro-crystalline cellulose and blends thereof; starch ethylenevinyl alcohols, polycyanoacrylates; polyphosphazenes; nonbioabsorbable polymers such as polyacrylate, polymethyl methacrylate, polytetrafluoroethylene, polyurethane and polyamide; etc. Preferred binders are polyhydroxybutyrate, polyhydroxyvalerate and tyrosine-based polycarbonates. When employed, binders will typically represent from about 5 to about 70 weight percent of the substrate bone particle-containing composition, calculated prior to compression of the substrate composition.

The binder acts as a matrix which binds the bone particles, thus providing coherency in a fluid environment and also improving the mechanical strength of the osteoimplant. Preferably, the binder is a cyanoacrylate binder. More preferably, the cyanoacrylate binder comprises ester chain, N-butyl, or butyl cyanoacrylates. Also, preferably the cyanoacrylate is a long chain cyanoacrylates.

Suitable fillers include graphite, pyrolytic carbon, bioceramics, bone powder, demineralized bone powder, anorganic bone (i.e., bone mineral only, with the organic constituents removed), dentin tooth enamel, aragonite, calcite, nacre, amorphous calcium phosphate, hydroxyapatite, tricalcium phosphate, Bioglass® and other calcium phosphate materials, calcium salts, etc. Preferred fillers are demineralized bone powder and hydroxyapatite. When employed, filler will typically represent from about 5 to about 80 weight percent of the substrate bone particle-containing composition, calculated prior to compression of the substrate composition.

Suitable fibers include carbon fibers, collagen fibers, tendon or ligament derived fibers, keratin, cellulose, hydroxyapatite and other calcium phosphate fibers. When employed, fiber will typically represent from about 5 to about 75 weight percent of the substrate bone particle-containing composition, calculated prior to compression of the substrate composition.

Additionally fibers may be utilized out of various components within the bone composite material to provide strength to the composite. Generally, the addition of fibers in materials assist in providing greater resistance to fractures or breakages due to tension placed upon the composite. For purposes of this patent application, fibers are generally defined as any materials having a greater length then width and may include a variety of organic and inorganic materials including straws and natural fibers as well as polymers, strings as well as carbon fibers. In further embodiments metallic fibers may also be utilized in providing greater strength and durability to the composite material.

Suitable plasticizers include liquid polyhydroxy compounds such as glycerol, monoacetin, diacetin, etc. Glycerol and aqueous solutions of glycerol are preferred. When employed, plasticizer will typically represent from about 20 to about 80 weight percent of the substrate bone particle-containing composition, calculated prior to compression of the substrate composition.

Suitable biostatic/biocidal agents include antibiotics such as erythromycin, bacitracin, neomycin, penicillin, polymycin B, tetracyclines, biomycin, chloromycetin, and streptomycins, cefazolin, ampicillin, azactam, tobramycin, clindamycin and gentamicin, povidone, sugars, mucopolysaccharides, etc. Preferred biostatic/biocidal agents are antibiotics. When employed, biostatic/biocidal agent will typically represent from about 10 to about 95 weight percent of the substrate bone particle-containing composition, calculated prior to compression of the substrate composition.

Suitable surface active agents include the biocompatible nonionic, cationic, anionic and amphoteric surfactants. Preferred surface active agents are the nonionic surfactants. When employed, surface active agent will typically represent from about 1 to about 80 weight percent of the substrate bone particle-containing composition, calculated prior to compression of the substrate composition.

Any of a variety of bioactive substances can be incorporated in, or associated with, the bone particles. Thus, one or more bioactive substances can be combined with the bone particles by soaking or immersing the bone particles in a solution or dispersion of the desired bioactive substance(s). Bioactive substances include physiologically or pharmacologically active substances that act locally or systemically in the host.

Bioactive substances which can be readily combined with the bone particles include, e.g., collagen, insoluble collagen derivatives, etc., and soluble solids and/or liquids dissolved therein; antiviricides, particularly those effective against HIV and hepatitis; antimicrobials and/or antibiotics such as erythromycin, bacitracin, neomycin, penicillin, polymycin B, tetracyclines, biomycin, chloromycetin, and streptomycins, cefazolin, ampicillin, azactam, tobramycin, clindamycin and gentamicin, etc.; biocidal/biostatic sugars such as dextran, glucose, etc.; amino acids; peptides; vitamins; inorganic elements; co-factors for protein synthesis; hormones; endocrine tissue or tissue fragments; synthesizers; enzymes such as collagenase, peptidases, oxidases, etc.; polymer cell scaffolds with parenchymal cells; angiogenic agents and polymeric carriers containing such agents; collagen lattices; antigenic agents; cytoskeletal agents; cartilage fragments; living cells such as chondrocytes, bone marrow cells, mesenchymal stem cells, natural extracts, genetically engineered living cells or otherwise modified living cells; DNA delivered by plasmid or viral vectors; tissue transplants; demineralized bone powder; autogenous tissues such as blood, serum, soft tissue, bone marrow, etc.; bioadhesives, bone morphogenic proteins (BMPs); osteoinductive factor; fibronectin (FN); endothelial cell growth factor (ECGF); cementum attachment extracts (CAE); ketanserin; human growth hormone (HGH); animal growth hormones; epidermal growth factor (EGF); interleukin-1 (IL-1); human alpha thrombin; transforming growth factor (TGF-beta); insulin-like growth factor (IGF-1); platelet derived growth factors (PDGF); fibroblast growth factors (FGF, bFGF, etc.); periodontal ligament chemotactic factor (PDLGF); somatotropin; bone digesters; antitumor agents; immuno-suppressants; permeation enhancers, e.g., fatty acid esters such as laureate, myristate and stearate monoesters of polyethylene glycol, enamine derivatives, alpha-keto aldehydes, etc.; and nucleic acids. Preferred bioactive substances are currently bone morphogenic proteins and DNA delivered by plasmid or viral vector. When employed, bioactive substance will typically represent from about 0.1 to about 20 weight percent of the substrate bone particle-containing composition, calculated prior to compression of the substrate composition.

It will be understood by those skilled in the art that the foregoing biocompatible components are not intended to be exhaustive and that other biocompatible components may be admixed with bone particles within the practice of the substrate composite.

The total amount of such optionally added biocompatible substances will typically range from about 0 to about 95% weight/volume (w/v), preferably from about 1 to about 60% w/v, more preferably from about 5 to about 50% w/v, weight percent of the substrate bone particle-containing composition, based on the weight of the entire composition prior to compression of the substrate composition, with optimum levels being readily determined in a specific case by routine experimentation.

One method of fabricating the substrate bone particle-containing composition which can be advantageously utilized herein involves wetting a quantity of bone particles, of which at least about 60 weight percent preferably constitute elongate bone particles, with a wetting agent as described above to form a composition having the consistency of a slurry or paste. Optionally, the wetting agent can comprise dissolved or admixed therein one or more biocompatible substances such as biocompatible binders, fillers, plasticizers, biostatic/biocidal agents, surface active agents, bioactive substances, etc., as previously described.

Preferred wetting agents for forming the slurry or paste of bone particles include water, liquid polyhydroxy compounds and their esters, and polyhydroxy compounds in combination with water and/or surface active agents, e.g., the Pluronics™ series of nonionic surfactants. Water is the most preferred wetting agent for utilization herein. The preferred polyhydroxy compounds possess up to about 12 carbon atoms and, where their esters are concerned, are preferably the monoesters and diesters. Specific polyhydroxy compounds of the foregoing type include glycerol and its monoesters and diesters derived from low molecular weight carboxylic acids, e.g., monoacetin and diacetin (respectively, glycerol monoacetate and glycerol diacetate), ethylene glycol, diethylene glycol, triethylene glycol, 1,2-propanediol, trimethylolethane, trimethylolpropane, pentaerythritol, sorbitol, and the like. Of these, glycerol is especially preferred as it improves the handling characteristics of the bone particles wetted therewith and is biocompatible and easily metabolized. Mixtures of polyhydroxy compounds or esters, e.g., sorbitol dissolved in glycerol, glycerol combined with monoacetin and/or diacetin, etc., are also useful. Where elongate bone particles are employed, some entanglement of the wet bone particles will result. Preferably, excess liquid can be removed from the slurry or paste, e.g., by applying the slurry or paste to a form such as a flat sheet, mesh screen or three-dimensional mold and draining away excess liquid.

Where, in a particular composition, the bone particles have a tendency to quickly or prematurely separate or to otherwise settle out from the slurry or paste such that application of a fairly homogeneous composition is rendered difficult or inconvenient, it can be advantageous to include within the composition a substance whose thixotropic characteristics prevent or reduce this tendency. Thus, e.g., where the wetting agent is water and/or glycerol and separation of bone particles occurs to an excessive extent where a particular application is concerned, a thickener such as a solution of polyvinyl alcohol, polyvinylpyrrolidone, cellulosic ester such as hydroxypropyl methylcellulose, carboxy methylcellulose, pectin, xanthan gum, food-grade texturizing agent, gelatin, dextran, collagen, starch, hydrolyzed polyacrylonitrile, hydrolyzed polyacrylamide, polyelectrolyte such as polyacrylic acid salt, hydrogels, chitosan, other materials that can suspend particles, etc., can be combined with the wetting agent in an amount sufficient to significantly improve the suspension-keeping characteristics of the composition.

The binder is added in an amount to sufficiently provide a cohesive ground substrate bone composite that can be used in skeletal repair and revisions methods without the ground bone coming apart. Preferably, the binder is present in an amount of from about 5% to about 80% w/v. More preferably, the binder may be present in a range of about 20% to about 66% w/v. More preferably, the binder may be present in an amount of from about 20 to about 50%. Another preferred range of binder is it being present in an amount of from about 15% to about 66% w/v.

Additionally, the particular binder used can be varied according to desired properties. For example, cyanoacrylates can be used as a binder in the production of cortical onlay plates and is preferably present in amount of from 20% to 30%. A binder may also be combined with at least one other binder. The binder is applied by injection, spray, bath, soaking or layering.

The above general ranges allow one of ordinary skill in the art to create a substrate composite of proper density and mechanical properties and further allows the same basic device to be tailored to individual patients and situations.

As stated above, the preferred binder is a biocompatible cyanoacrylate. Preferred biocompatible cyanoacrylates include ester chain, N-butyl, and butyl cyanoacrylates. When a cyanoacrylate binder is used, a preferred amount is from about 5 to about 80%, preferably from about 20 to about 66%, more preferably from about 20 to about 50%. The cyanoacrylate binder may be combined with at least one other binder. More specifically, the cyanoacrylate binder described herein may also be a cyanoacrylate-comprising binder.

Additionally, through the use of the cyanoacrylate binder the composite is significantly easier to install and provides for less down time and greater curing qualities than previous prior art implants. Generally, cyanoacrylate polymerizes in the presence of moisture and as such through the addition of moisture to the composite, either previously to installing the implant containing the composite material or through moisture inherent within the patient, the cyanoacrylate will begin to polymerize. Generally this process takes less than about thirty minutes for the cyanoacrylate to polymerize by at least 50% thus providing for a bone composite material with growth accelerators that sets exceptionally fast within a patient. The fast setting rate is significant as generally, other implants may take on the order of hours to set requiring significant immobility of the patient during the setting procedure.

In addition to the materials described above, at least one other adhesive substance can optionally be used as a matrix to form a substrate composite bone material (in combination with or without at least one cyanoacrylate). For example, fibrin is a substance formed by human blood when it clots. Fibrin bonds the platelets together in the formation of, e.g., clots and scabs. Alternatively, fibrin glue can be manufactured. Other biocompatible adhesives can also be used. In addition, there exist a number of biocompatible gels which can be used as a matrix adhesive for holding bone powder together.

The vacuum force applied to the mold typically ranges from about 29.9 inches of Hg to about 19.7 inches (based on a standard barometer reading of 29.92 inches of Hg at atmospheric pressure being 0% vacuum. Preferably, the vacuum force is about 29.5 inches Hg to about 24 inches Hg. Typically, the vacuum force is about 28 inches Hg.

Preferably, the vacuum force is applied simultaneous with the injection or spraying of binder. The vacuum force helps distribute the binder throughout the ground bone tissue.

The vacuum forces applied for a period of from about one second to about one hour upon the substrate bone composite prior to the introduction of the carrier with growth accelerator.

Additionally, pressure during the formation of the substrate bone composite can be tailored to the desired outcome and structural properties of the composite. The pressures can range from about 14.7 psi to less than 10,000 psi. Generally, lower pressures from a range of about 14.7 psi to about 100 psi can be used to form bone composites useful for skeletal repair and revision. Higher pressures of from about 100 psi to less than 1000 psi can be utilized to form substrate composites which may be eventually used for screws and other load-bearing tasks.

Generally, the substrate bone composite remains within the mold under pressure for a greater length of time then prior art bone composites as the longer duration of times allows for the polymerization of n-Butyl cyanoacrylate within the lattice work of structural and the adhering cyanoacrylate polymer beads. Generally, this will provide non-cyanoacrylate enveloped locations on each particle of the demineralized bone matrix of the substrate bone composites. Advantageously, these open voids can provide for greater exposure of bone morphogenic proteins and thereby allow osteoinductivity to take place.

After the longer duration of vacuum applied to the substrate bone composite, the substrate bone composite may then be subjected to an injection of carrier including the growth accelerator. As dependent upon the carrier, the values of viscosity, weight, and volume of the carrier and growth accelerator combination can be tailored so that the injection of the carrier and the growth accelerator may better permeate the substrate bone composition.

Immediately prior to the application of the carrier and growth accelerator, the substrate bone composite may be injected with water, preferably non-isotonic water with a predetermined pH factor. A vacuum may be applied for a short time with this step preferably hydrating the bone particles of a substrate bone composite prior to the addition of the carrier-growth accelerator combination.

In embodiments wherein the bone particles of the substrate bone composites are hydrated prior to the application of the carrier with growth accelerator, the carrier and growth accelerator are then applied to the substrate bone composite in a predetermined amount. Preferably, the carrier is a bioabsorbable cyanoacrylate as previously discussed and may include a TGF which both are combined and the injected into the substrate bone composite with the application of pressure so that the fluid injection of the carrier and growth accelerator permeate into the substrate bone composite. After a predetermined amount of time, the bone composite containing the carrier and growth accelerator may be removed from the mold with the composite removed. Following the removal from the mold, the improved composite may be shaped into the desired product. Alternatively, if the mold utilized is shaped as a desired product, the improved composite may be inspected for any out-of-tolerance, measurement, or shape. Differences can be corrected in any number of ways including with a light file, grinding, or milling. Following, the improved composite can be sterilized and packaged.

Advantageously, the utilization of the carrier with growth factor may increase the build up and production of bone cells at the localized site of the implant within the patient. In a preferable arrangement through the use of a bioabsorbable carrier and growth factor such as TGF, the bioabsorbable carrier may be absorbed by the body of the patient and the growth factor released from the improved bone composite, which subsequently induces an accelerated and improved structural bone fusion with the improved bone composite implant.

As such, the improved bone composite implant of the present invention has improved osteoinductivity characteristic whereby upon implantation a growth accelerator such as a TGF may emanate from the improved bone composite upon implantation, thus providing for improved bone formation with an even less invasive surgical procedure.

In additional embodiments of the present invention, the carrier with growth accelerator may include a predetermined ionic or magnetic charge so that the growth factor may more readily mix with bone-generating components of the patient's body, and thus, increase the rate of bone formation at an even greater rate. This may include the use of a carrier with growth accelerator having either a positive or negative magnetic charge which may be predetermined prior to implantation with regard to whether the human body possesses either positive or negative magnetic polarity. Advantageously, the improved bone composite, including a carrier and growth accelerator with a magnetic charge, when formed in this manner and implanted into a patient's body, the patient's own plasma maybe attracted to this polarized carrier and growth accelerator, thus resulting in a healing rate of the patient to be accelerated faster than realized in the prior art.

Furthermore, the improved bone composite may optionally also undergo crosslinking as described additionally in the U.S. Pat. No. 7,001,551 and is further described in U.S. Pat. No. 6,294,187 which is hereby incorporated by reference in its entirety so as to improve the strength of the improved bone composite.

Such crosslinking of the bone containing composition though not required for the use of the improved bone composite, can be effected by a variety of known methods including chemical reaction, the application of energy such as radiant energy, which includes irradiation by UV light or microwave energy, drying and/or heating and dye-mediated photo-oxidation; dehydrothermal treatment in which water is slowly removed while the bone particles are subjected to a vacuum; and, enzymatic treatment to form chemical linkages at any collagen-collagen interface. The preferred method of forming chemical linkages is by chemical reaction.

Chemical crosslinking agents include those that contain bifunctional or multifunctional reactive groups, and which react with surface-exposed collagen of adjacent bone particles within the bone particle-containing composition. By reacting with multiple functional groups on the same or different collagen molecules, the chemical crosslinking agent increases the mechanical strength of the osteoimplant.

Chemical crosslinking involves exposing the bone particles presenting surface-exposed collagen to the chemical crosslinking agent, either by contacting bone particles with a solution of the chemical crosslinking agent, or by exposing bone particles to the vapors of the chemical crosslinking agent under conditions appropriate for the particular type of crosslinking reaction. For example, the osteoimplant of this invention can be immersed in a solution of crosslinking agent for a period of time sufficient to allow complete penetration of the solution into the osteoimplant. Crosslinking conditions include an appropriate pH and temperature, and times ranging from minutes to days, depending upon the level of crosslinking desired, and the activity of the chemical crosslinking agent. The resulting osteoimplant is then washed to remove all leachable traces of the chemical.

Suitable chemical crosslinking agents include mono- and dialdehydes, including glutaraldehyde and formaldehyde; polyepoxy compounds such as glycerol polyglycidyl ethers, polyethylene glycol diglycidyl ethers and other polyepoxy and diepoxy glycidyl ethers; tanning agents including polyvalent metallic oxides such as titanium dioxide, chromium dioxide, aluminum dioxide, zirconium salt, as well as organic tannins and other phenolic oxides derived from plants; chemicals for esterification or carboxyl groups followed by reaction with hydrazide to form activated acyl azide functionalities in the collagen; dicyclohexyl carbodiimide and its derivatives as well as other heterobifunctional crosslinking agents; hexamethylene diisocyante; sugars, including glucose, will also crosslink collagen.

Glutaraldehyde crosslinked biomaterials have a tendency to over-calcify in the body. In this situation, should it be deemed necessary, calcification-controlling agents can be used with aldehyde crosslinking agents. These calcification-controlling agents include dimethyl sulfoxide (DMSO), surfactants, diphosphonates, aminooleic acid, and metallic ions, for example ions of iron and aluminum. The concentrations of these calcification-controlling agents can be determined by routine experimentation by those skilled in the art.

When enzymatic treatment is employed, useful enzymes include those known in the art which are capable of catalyzing crosslinking reactions on proteins or peptides, preferably collagen molecules, e.g., transglutaminase as described in Jurgensen et al., The Journal of Bone and Joint Surgery, 79-a (2), 185 193 (1997).

Formation of chemical linkages can also be accomplished by the application of energy. One way to form chemical linkages by application of energy is to use methods known to form highly reactive oxygen ions generated from atmospheric gas, which in turn, promote oxygen crosslinks between surface-exposed collagen. Such methods include using energy in the form of ultraviolet light, microwave energy and the like. Another method utilizing the application of energy is a process known as dye-mediated photo-oxidation in which a chemical dye under the action of visible light is used to crosslink surface-exposed collagen.

Another method for the formation of chemical linkages is by dehydrothermal treatment which uses combined heat and the slow removal of water, preferably under vacuum, to achieve crosslinking of bone particles. The process involves chemically combining a hydroxy group from a functional group of one collagen molecule and a hydrogen ion from a functional group of another collagen molecule reacting to form water which is then removed resulting in the formation of a bond between the collagen molecules.

Furthermore, as previously discussed the substrate bone composite as described in U.S. Pat. No. 7,001,551 is the preferred type of substrate composite as it comprises random voids present at both the surface as well as the interior of the composite. Generally, the voids or spaces of this preferred substrate composite vary in size and shape and have a width of up to about 1000 microns. However, a variety of other different bone composite substrates may be utilized so long as the incorporation of a growth factor thereinto is possible. Furthermore, while preferably a carrier and more preferably a bioabsorbable carrier is utilized, the growth accelerated may be integrated within the substrate bone composite without a carrier.

Accordingly, by the practice of the present invention, an improved bone composite and method of making the improved bone composite having heretofore unrecognized characteristics is prepared. These bone composites exhibit exceptional induction of bone formation upon implantation within a patient.

The disclosure of all cited patents and publications referred to in this application are incorporated herein by reference.

The above description is intended to enable the person skilled in the art to practice the invention. It is not intended to detail all the possible variations and modification that are apparent to the skilled worker upon reading the description.

What is claimed is:

1. A method of forming a bone composite with a growth accelerator, comprising:
a) providing ground bone tissue;
b) molding the ground bone tissue into a bone composite; and
c) adding a growth accelerator to the bone composite to form a bone composite with a growth accelerator.

2. The method of claim 1 further comprising obtaining bone tissue and grinding the bone tissue to form ground bone tissue prior to step a).

3. The method of claim 1 wherein step c) further comprises adding the growth accelerator with a carrier to the bone composite.

4. The method of claim 3 wherein the carrier comprises a binder.

5. The method of claim 4 wherein the binder comprises cyanoacrylate.

6. The method of claim 1 wherein the growth accelerator comprises at least one transforming growth factor.

7. The method of claim 1 wherein the growth accelerator comprises at least one bone morphogenetic protein.

8. The method of claim 7 wherein the bone morphogenetic protein is selected from the group consisting of BMP-2, BMP-3, BMP-4, BMP-7, BMP-8a and combinations thereof.

9. The method of claim 1 wherein the growth accelerator comprises one or more hormones.

10. The method of claim 9 wherein the one or more hormones comprise a synthetic hormone.

11. The method of claim 9 wherein the one or more hormones comprise recombinantly created hormones.

12. The method of claim 9 wherein the one or more hormones comprise amylin.

13. The method of claim 9 wherein the one or more hormones comprise adrenonedullin.

14. The method of claim 1 wherein the growth accelerator comprises a parathyroid hormone-affecting compound.

15. A bone composite, comprising:
ground bone tissue including cortical bone tissue;
a growth accelerator applied to the ground bone tissue to increase bone fusion; and
a binder applied to the ground bone tissue to provide coherency to the bone composite.

16. The composite of claim 15 wherein the growth accelerator comprise one or more transforming growth factors.

17. The composite of claim 15 wherein the growth accelerator comprises one or more bone morphogenetic proteins.

18. The composite of claim 15 wherein the growth accelerator comprises one or more hormones.

19. The composite of claim 18 wherein the one or more hormones comprise synthetic hormones.

20. The composite of claim 18 wherein the one or more hormones comprise calcitonin.

21. The composite of claim 18 wherein the one or more hormones comprise parathyroid hormone.

22. The composite of claim 18 wherein the one or more hormones comprise amylin.

23. The composite of claim 18 wherein the one or more hormones comprise adrenonedullin.

24. The composite of claim 15 wherein the growth accelerator comprises a parathyroid-affecting compound.

25. The composite of claim 15 wherein the growth accelerator comprises a calcitonin-affecting compound.

26. The composite of claim 15 wherein the binder comprises a cyanoacrylate binder.

27. The composite of claim 15 further comprising strengthening elements dispersed within the composition.

28. The composition of claim 27 wherein the strengthening elements have a greater length than width.

29. The composition of claim 28 wherein the strengthening elements comprise fibers.

30. A method of implanting a bone composite with a growth accelerator into a mammal, comprising:
a) providing ground bone tissue, a growth accelerator and a binder;
b) mixing the bone tissue, the growth accelerator, and the binder to form a bone composite with a growth accelerator;
c) inserting the bone composite with the growth accelerator into the mammal;
d) setting the binder of the bone composite with the growth accelerator to provide coherency to the composite.

31. The method of claim 30 wherein the binder comprises a cyanoacrylate binder.

32. The method of claim 31 wherein in step d) the setting of the binder comprises polymerization of the cyanoacrylate binder by at least 50% in less than about 30 minutes after being inserted into the mammal.
